(19) United States
(12) Patent Application Publication
(10) Pub. No.: US 2017/0356869 A1
(43) Pub. Date: Dec. 14, 2017

(54) GAS SENSOR, HUMIDITY SENSOR, AND METHOD FOR FORMING A SENSOR LAYER

(71) Applicant: Infineon Technologies AG, Neubiberg (DE)

(72) Inventors: Matthias Koenig, Freising (DE); Florian Bachl, Regensburg (DE); Alexander Zoepfl, Regensburg (DE); Guenther Ruhl, Regensburg (DE)

(21) Appl. No.: 15/620,868

(22) Filed: Jun. 13, 2017

(30) Foreign Application Priority Data
Jun. 13, 2016 (DE) 10 2016 110 786.7

(51) Int. Cl. G01N 27/12; G01N 33/00

(52) U.S. Cl. CPC G01N 27/121; G01N 27/127; G01N 27/123; G01N 27/125; G01N 33/0027

(57) ABSTRACT

Various embodiments relate to a gas sensor, including: a carrier, an electrode structure; and a sensor layer in contact with the electrode structure, wherein the sensor layer includes or essentially consists of turbostratic graphite.

### CROSS-REFERENCE TO RELATED APPLICATION

This application claims priority to German Patent Application Serial No. 10 2016 110 786.7, which was filed Jun. 13, 2016, and is incorporated herein by reference in its entirety.

### TECHNICAL FIELD

Various embodiments relate generally to a gas sensor, a humidity sensor, and a method for forming a sensor layer.

### BACKGROUND

Hygrometers, i.e. instruments used for measuring the moisture content in the atmosphere, can be used in various fields of application, e.g. in climate home stations, automotive, air conditioning, smart phones, farming, sports, process control in industry, and the like. Currently, there are a various sensor concepts available, but actually most common sensors are based on the principle of capacitive or resistive hygrometers. In such types of hygrometers, an active sensor material may be used, typically a polymer that changes its resistance or capacitance dependent on the humidity in the environment. The change of resistance or capacitance may be caused by a change of the dielectric constant of the active sensor material, by a change of the thickness of the active sensor material, or by a change of another physical property of the active sensor material. These changes can be electrically registered and evaluated by an electronic circuit, providing for example a readout voltage that is proportional to the ambient humidity. In general, a sensor may include an active sensor material that changes its physical properties as a result of an exposure to a material to be sensed, whereby the changes of the physical properties can be electrically registered and evaluated. The sensor may sense a material in liquid or in gaseous state, generally referred to as a fluid.

### SUMMARY

According to various embodiments, a sensor, e.g. a fluid sensor, may include a carrier, an electrode structure; and a sensor layer in contact with the electrode structure, wherein the sensor layer includes or essentially consists of turbostratic graphite.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 schematically shows a ternary phase diagram including sp²-hybridized carbon, sp³-hybridized carbon, and hydrogen;

FIG. 2 shows a schematic flow diagram of a method for forming a sensor layer, according to various embodiments;

FIG. 3A and FIG. 3B respectively show a sensor layer of a sensor in a schematic cross sectional view, according to various embodiments;

FIGS. 4A to 4C respectively show a sensor in a schematic cross sectional view, according to various embodiments;

FIG. 5 shows a sensor in a schematic cross sectional view, according to various embodiments;

FIG. 6 shows an electron microscopy image of a surface of a sensor layer, according to various embodiments;

FIG. 7A shows an image of a sensor in a top view, according to various embodiments;

FIG. 7B shows a measurement of a drift resistance dependent on a voltage applied to a sensor layer, according to various embodiments;

FIG. 8 shows a measurement of a resistance dependent on a controlled exposure to humidity, according to various embodiments;

FIG. 9A and FIG. 9B respectively show a measurement of a characteristic property of a sensor, according to various embodiments;

FIG. 9C shows a measurement of a characteristic property of a sensor, according to various embodiments;

FIG. 10 shows an electron microscopy image of a surface of a sensor layer, according to various embodiments; and

FIG. 11A and FIG. 11B respectively show a measurement of a resistance dependent on a controlled exposure to carbon monoxide, according to various embodiments.

### DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments. Various embodiments are described in connection with methods and various embodiments are described in connection with devices. However, it may be understood that embodiments described in connection with methods may similarly apply to the devices, and vice versa.

The terms “at least one” and “one or more” may be understood to include any integer number greater than or equal to one, i.e. one, two, three, four, [ . . . ], etc. The term “a plurality” may be understood to include any integer number greater than or equal to two, i.e. two, three, four, five, [ . . . ], etc.

The word “over”, used herein to describe forming a feature, e.g. a layer “over” a side or surface, may be used to mean that the feature, e.g. the layer, may be formed “directly on”, e.g. in direct contact with, the implied side or surface. The word “over”, used herein to describe forming a feature, e.g. a layer “over” a side or surface, may be used to mean that the feature, e.g. the layer, may be formed “indirectly on” the implied side or surface with one or more additional layers being arranged between the implied side or surface and the formed layer.

In like manner, the word “cover”, used herein to describe a feature disposed over another, e.g. a layer “covering” a side or surface, may be used to mean that the feature, e.g. the layer, may be disposed over, and in direct contact with, the implied side or surface. The word “cover”, used herein to describe a feature disposed over another, e.g. a layer “covering” a side or surface, may be used to mean that the feature, e.g. the layer, may be disposed over, and in indirect contact with, the implied side or surface with one or more additional layers being arranged between the implied side or surface and the covering layer.

The term “lateral” used with regards to the “lateral” extension of a structure (or of a structure element) provided on or in a carrier (e.g. a layer, a substrate, a wafer, or a semiconductor work piece) or “laterally” next to, may be used herein to mean an extension or a positional relationship along a surface of the carrier. That means that a surface of a carrier (e.g. a surface of a substrate, a surface of a wafer, or a surface of a work piece) may serve as reference, commonly referred to as the main processing surface. Further, the term “width” used with regards to a “width” of a structure (or of a structure element) may be used herein to mean the lateral extension of a structure. Further, the term “height” used with regards to a height of a structure (or of a structure element), may be used herein to mean an extension of a structure along a direction perpendicular to the surface of a carrier (e.g. perpendicular to the main processing surface of a carrier). The term “thickness” used with regards to a “thickness” of a layer may be used herein to mean the spatial extension of the layer perpendicular to the surface of the support (the material or material structure) on which the layer is deposited. If a surface of the support is parallel to the surface of the carrier (e.g. parallel to the main processing surface) the “thickness” of the layer deposited on the surface of the support may be the same as the height of the layer.

Essentially three types of materials are used in semiconductor industry for manufacturing an electronic device (e.g. a sensor), namely electrically insulating (that means electrically non-conductive) materials, electrically semiconductive materials, and electrically conductive materials.

Semiconductive materials have a moderate electrical conductivity, e.g. an electrical conductivity in the range from about 10⁻⁶ Siemens per meter (S/m) to about 10⁶ S/m. Further, semiconductive materials have a typical band gap between the valence and conduction bands that results in a negative temperature coefficient of the electrical resistivity. Intrinsic semiconductive materials may include single-element semiconductors (e.g. silicon, germanium, etc.), compound semiconductors (e.g. gallium arsenide, silicon carbide, etc.), and organic semiconductors (e.g. polythiophen, pentacen, etc.). The electrical properties of intrinsic semiconductive materials may be modified by doping these materials, e.g. p-type or n-type. Lightly and moderately doped semiconductors are referred to as extrinsic semiconductors. However, a semiconductor may be doped to such high level that it is similar to a conductor (referred to as a degenerate semiconductor or degenerate doping).

Electrically conductive materials have a sufficiently high electrical conductivity to substantially contribute to current transport, e.g. an electrical conductivity greater than about 10⁶ S/m, e.g. greater than about 10⁷ S/m. Electrically conductive materials may include single-element conductors, e.g. metals as for example aluminum, copper, silver, gold, tantalum, titanium, etc., compounds or alloys, e.g. metal nitrides like titanium nitride, tantalum nitride, etc., or metal alloys like aluminum/copper, and the like.

Electrically insulating materials have a low electrical conductivity, e.g. an electrical conductivity less than about 10⁻⁶ S/m, e.g. less than about 10⁻¹⁰ S/m. Electrically insulating materials may include oxides, e.g. silicon oxide or metal oxides like aluminum oxide, and the like. Electrically insulating materials may further include polymers, e.g. polyimide, and the like. Electrically insulating materials may include high-k (with k greater than 4.2) dielectrics, e.g. for forming a gate structure of a field effect transistor, or low-k dielectrics (with k lower than 4.2), e.g. as inter-metal dielectric of a metallization structure.

Conventionally, gas sensors may include a sensor layer of a receptively suited material, e.g. a graphene sheet may be used as sensor layer. However, graphene may have some severe manufacturing issues, for which solutions may be required. Many different materials are known as sensitive materials for gas sensors and humidity sensors, e.g. metal oxides, polymers (e.g. dielectrics polymers for capacitive measurements), salts, conductive polymers (e.g. for resistive measurements). Most of these materials have to be heated to remove the water or gas after absorption. However, there may be aging effects due to aggressive ambient gases. The most widely used materials for humidity sensors are organic polymers, which may not be sufficiently long-term stable due to degradation in ambient atmosphere or ambient gases. Further, capacitive measurement principles may need a measurement periphery with some complexity. Resistive humidity sensors may be based on amorphous carbon, wherein the amorphous carbon may be deposited conventionally via a hot filament physical vapor deposition technique. Since this may not be a standard deposition technique in semiconductor manufacturing, there may be high costs to manufacture such layers at industrial scale. Further, a physical vapor deposition of amorphous carbon may cause problems due to particle generation, since the generated particles may introduce an undesired defect density into the deposited layer and, therefore, devices manufactured using conventional physical vapor deposition of amorphous carbon may not be long term stable or may be expensive to manufacture.

Graphene is a two-dimensional, atomic-scale, honeycomb sheet of carbon. For manufacturing of a graphene-based gas sensors a single-layer graphene sheet has to be prepared, which may require several complex process steps, for example: first, a chemical vapor deposition (CVD) carried out at high temperatures (e.g. in the range from about 800° C. to about 1000° C.) on a metallic substrate; and, subsequent, a transfer step to a dielectric substrate. This processing that includes the transfer of the graphene sheet may introduce wrinkles, holes, and particles into the graphene sheet that may lead to a degradation of its properties. Thus, a reproducible deposition technique for high-quality graphene sheets may not be available or may not be cost efficient. Forming a graphene sheet by aggregating graphene flakes from a suspension may be possible in principle. However, this method may have a low quality and a poor reproducibility.

Carbon can be a basis for a variety of materials including materials based for example on pure carbon or on carbon compounds. The most commonly known modifications (also referred to as allotropes) of carbon are the diamond modification, the graphite modification, and molecule-like modifications, as for example graphene, graphane, fullerene, nanotubes, and the like. Further, carbon may form a vast number of different materials in combination with hydrogen. Carbon atoms may be bound together in different ways that are substantially described using the hybridization concept of mixing atomic orbitals, in this case s-orbital and p-orbital, into new hybrid orbitals that are sp-, sp2-, or sp3-orbitals.

In general, an amorphous material (for example amorphous carbon (a-C), hydrogenated amorphous carbon (a-C:H), tetrahedral amorphous carbon (ta-C), and hydrogenated tetrahedral amorphous carbon (ta-C:H)) does not have a long range ordered crystalline structure. Amorphous carbon may include carbon atoms connected to adjacent carbon atoms and/or hydrogen atoms (in a short range order) forming either an sp²-hybridized bonding structure (three sp²-orbitals are oriented in a plane symmetrical to each other (with a trigonal symmetry)) or an sp³-hybridized bonding structure (four sp³-orbitals are tetrahedrally aligned equiangularly to each other). However, also the short-range order of amorphous carbon may be disturbed, e.g. the C-rings may be “warped” or disordered, which may have an impact to the Raman spectra (e.g. a D-peak, or a peak broadening). Amorphous carbon may be electrically insulating.

Generally, amorphous carbon layers may be formed via plasma-enhanced chemical vapor deposition (PECVD) or physical vapor deposition (PVD), also referred to as amorphous carbon films formed by thin film deposition. Therefore, conventionally, amorphous carbon layers may be deposited via deposition processes under non-equilibrium conditions that are kinetically controlled. The chemical and physical properties of an amorphous carbon layer may depend on the used deposition technique and/or the applied deposition conditions. Tuning parameters for the deposition may be the deposition temperature, the source material, the pressure, and the like. Various carbon based materials, as for example amorphous carbon or hydrogenated amorphous carbon, may be classified in a ternary phase diagram based on the respective sp²-sp³ content of the material and the hydrogen content of the material.

A content of a constituent in a mixture may be expressed by the molar fraction or molar percentage (abbreviated, mol-%), substantially based on the number of atoms of the respective materials. The molar percentage may be conversed to the equivalent atomic percentage (abbreviated, at-%) using the Avogadro constant. However, a content of a constituent in a mixture may also be described as mass fraction or percentage by mass (abbreviated, wt-%,). A conversion of mass fraction and molar fraction may be possible using the respective molar mass of the materials.

A concentration of a specific material in a mixture may be expressed by mass concentration, molar concentration, or number of atoms concentration. The molar concentration is the amount of a constituent (in moles) divided by the volume of the mixture. Using the molar mass of the constituent and/or the Avogadro constant a conversion of the mass concentration, the molar concentration, and the number of atoms concentration into each other is possible. The concentration may be also expressed in parts per million (abbreviated, ppm).

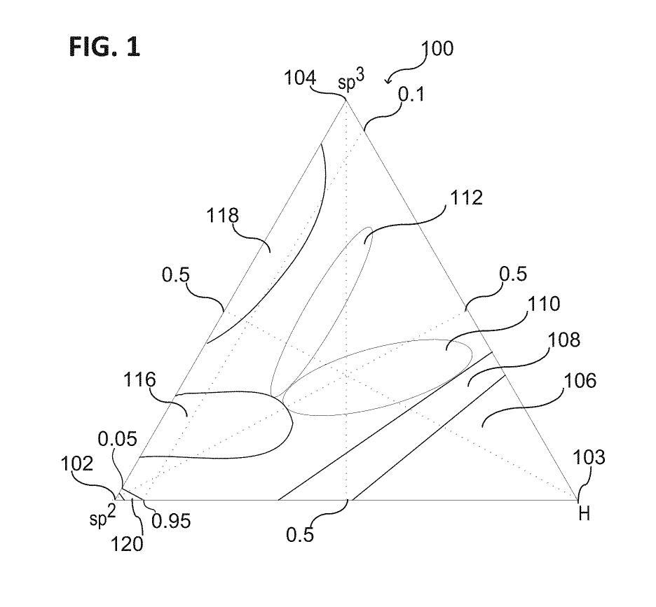

FIG. 1 shows a ternary phase diagram 100 including various modifications of carbon that can be deposited as layers using PVD or CVD, e.g. PECVD. The ternary phase diagram 100 illustrates a molar fraction of sp²-hybridized carbon, sp³-hybridized carbon, and hydrogen. Pure carbon phases are represented by two of the corners of the ternary phase diagram 100, that are the sp²-hybridized carbon 102 (e.g. the graphite phase of carbon) and the sp³-hybridized carbon 104 (e.g. the diamond phase of carbon). The third corner of the ternary phase diagram 100 represents hydrogen 103. The three outer lines represent the two-dimensional phase diagrams of the respective two components sp²-hybridized and sp³-hybridized carbon; sp³-hybridized carbon and hydrogen; and sp²-hybridized carbon and hydrogen.

Besides a region 106 of compositions forming no layers or being not accessible by means of layering processes (also referred to as thin film deposition processes, as for example PVD and/or CVD), the ternary phase diagram 100 illustrates various phases, as for example: hydrocarbon polymers 108; hydrogenated amorphous carbon (a-C:H) 110; sputtered amorphous carbon (a-C) and sputtered hydrogenated amorphous carbon (a-C:H) 116 (i.e. amorphous carbon formed by sputter deposition, e.g. using magnetron sputtering); hydrogenated tetrahedral amorphous carbon (ta-C:H) 112; and tetrahedral amorphous carbon 118 (ta-C).

As illustrated in and described with reference to FIG. 1, hydrocarbon polymers 108 may include a vast number of materials, as for example polyethylene (PE), polyacetylene (PAC), a plurality of polycyclic aromatic hydrocarbons, and the like. Hydrocarbon polymers 108 may include for example a molar percentage of hydrogen in the range from about 35% to about 65%. Further, the hydrocarbon polymers 108 may be for example completely sp³-hybridized, completely sp²-hybridized, or may include various mixtures of sp²-sp³-hybridized carbon atoms.

Hydrogenated amorphous carbon 110 (a-C:H); sputtered hydrogenated amorphous carbon 116 (a-C:H), and hydrogenated tetrahedral amorphous carbon 112 (ta-C:H) may include for example a molar percentage of hydrogen in the range from about 0% to about 60%. Hydrogen free amorphous carbon 116 (a-C) may be formed only by sputter deposition, e.g. by magnetron sputtering from a pure carbon source. However, PECVD processes may be used to form hydrogenated amorphous carbon in various chemical compositions with a molar percentage of sp³-hybridized carbon in the range from about 20% to about 65%.

The sp²-sp³ content of a layer may be evaluated for example based on their visible and ultraviolet (UV) Raman spectra. Further, the evaluation of the structural class of the amorphous carbon layers (e.g. a-C or a-C:H) may be possible via their ultraviolet (UV) Raman spectra, e.g. by the presence of the so-called T-peak in their UV-Raman spectra. Further, methods to identify the properties of a layer may be X-ray photoelectron spectroscopy (XPS) or electron energy loss spectroscopy (EELS) for the bonding state of carbon atoms; and high-resolution X-ray diffraction as well as high-resolution transmission electron microscopy (HRTEM) for the crystallographic structure. The hydrogen content of a material layer may be evaluated for example based Rutherford Backscattering (RBS) and/or Elastic Recoil Detection (ERD). The thickness of a material layer may be evaluated for example Scanning Electron Microscopy (SEM), e.g. by imaging a cross section of the material layer.

Chemical and/or physical properties of the respective material, for example optical properties, the band structure, electrical conductivity, and robustness towards chemically reactive materials, may depend on the respectively used deposition method or in other words on the respective position in the phase diagram represented by the hydrogen content and the sp²-sp³ content.

Various embodiments relate to a layer including or essentially consisting of turbostratic graphite. This layer may be a pure turbostratic graphite layer or may include at least 95 mol-% of turbostratic graphite. Turbostratic graphite may include a ratio of sp² hybridization to sp³ hybridization greater than about 95%. The ratio of sp² hybridization can be determined based on the amount of sp² hybridized carbon (Cₛₚ₂) divided by the total amount of both sp² and sp³ hybridized carbon (Cₛₚ₂+Cₛₚ₃). Accordingly, the ratio of sp³ hybridization, Cₛₚ₃/(Cₛₚ₂+Cₛₚ₃), of turbostratic graphite is less than about 5%. Further, according to various embodiments, turbostratic graphite may include hydrogen. Based on the ratio of sp² hybridization to sp³ hybridization and known hydrogen content, the corresponding molar fraction may be evaluated with reference to the ternary phase diagram, see FIG. 1. Accordingly, for low hydrogen content, e.g. less than about 10 mol-%, a corresponding molar percentage of sp³-hybridized carbon may be in the range from about 1% to about 5% and a corresponding molar percentage of sp²-hybridized carbon may be in the range from about 95% to about 99%. The molar percentage of hydrogen included in the turbostratic graphite may be greater than about 1%, e.g. in the range from about 1% to about 10%. With reference to the ternary phase diagram illustrated in FIG. 1, the turbostratic graphite may be represented by the region 120. Illustratively, turbostratic graphite 120 may not be regarded as pure graphite 102, cf. FIG. 1.

Further, according to various embodiments, turbostratic graphite may be polycrystalline. The crystallites of turbostratic graphite may have an average size of less than about 1 μm, e.g. in the range from about 1 nm to about 100 nm.

According to various embodiments, turbostratic graphite may be temperature stable up to about 2000° C. Above this temperature, turbostratic graphite 120 may crystallize to single crystalline graphite 102.

Graphite has a lamellae structure of stacked planar sheets, where each sheet is composed of hexagonally arranged carbon rings, illustratively in form of a honeycomb lattice. The distance between adjacent carbon atoms is 142 pm within the respective ring. The respectively adjacent planar sheets of a graphite stack may have two arrangements relative to each other; a hexagonal arrangement and a rhombohedral arrangement. The hexagonal graphite has an AB/AB/AB stacking sequence. The rhombohedral graphite has an ABC/ABC stacking sequence. In both cases the stacking distance between sheets is 335.4 pm.

According to various embodiments, turbostratic graphite may have a crystal structure in which the planar sheets have slipped out of alignment. According to various embodiments, two adjacent planar sheets may be rotationally misaligned along an axis perpendicular to the planar sheets. The rotational misalignment may be in the angular range from about 5° to about 25°. The rotational misalignment may be identified via structural techniques, as for example, transmission electron microscopy (TEM), scanning tunneling microscopy (STM), atomic force microscopy (AFM), e.g. using analysis of a Moiree pattern, and/or x-ray structure analysis.

According to various embodiments, turbostratic graphite may not have an ideal crystal structure, but rather a degree of disorder (also referred to as turbostratic disorder or rotational disorder). Therefore, the turbostratic graphite may have a c-axis lattice parameter (i.e. the distance of two directly adjacent lattice planes from each other) that is greater than the c-axis lattice parameter of graphite, e.g. greater than 0.335 nm; e.g. in the range from about 0.338 nm to about 0.350 nm, e.g. in the range from about 0.342 nm to about 0.346 nm, e.g. a c-axis lattice parameter of 0.344 nm. Illustratively, turbostratic graphite may include crystallites including graphene-like sheets that are stacked with rotational disorder.

According to various embodiments, turbostratic graphite may have an electrical conductivity that is greater than the electrical conductivity of amorphous carbon. According to various embodiments, a layer of turbostratic graphite may have a thickness greater than about 1 nm, e.g. in the range from about 1 nm to about 100 nm, e.g. in the range from about 1 nm to about 40 nm, or in the range from about 3 nm to about 40 nm. However, the layer of turbostratic graphite may also have a thickness greater than about 100 nm.

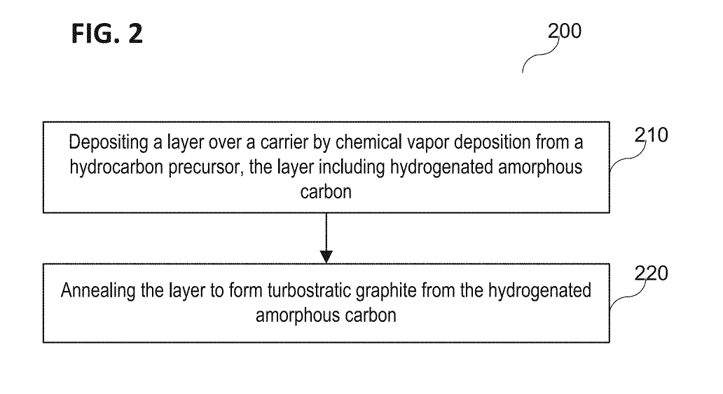

FIG. 2 illustrates a schematic flow diagram of a method 200 for forming a sensor layer, according to various embodiments. The method 200 may include, in 210, depositing a layer over a carrier by plasma enhanced chemical vapor deposition from a hydrocarbon precursor, the layer including hydrogenated amorphous carbon; and, in 220, annealing the layer to form turbostratic graphite from the hydrogenated amorphous carbon.

According to various embodiments, the chemical vapor deposition process (CVD) that may be used for step 210 of method 200 may be or may include a variety of modifications, as for example atmospheric pressure CVD (APCVD), sub-atmospheric pressure CVD (SAPCVD), low pressure CVD (LPCVD), ultrahigh vacuum CVD (UHVCVD), plasma enhanced CVD (PECVD), high density plasma CVD (HDPCVD), remote plasma enhanced CVD (RPECVD), atomic layer CVD (ALCVD), vapor phase epitaxy (VPE), metal organic CVD (MOCVD), hybrid physical CVD (HPCVD), and the like. Using for example APCVD, SAPCVD, LPCVD, UHVCVD, ALCVD, VPE MOCVD, or HPCVD turbostratic graphite may be formed if the deposition temperature is selected suitably high, e.g. equal to or greater than about 700° C. Using for example PECVD the deposited graphite layer may be disturbed due to the ion bombardment during deposition and may relax partially by forming sp³-domains. The PECVD process may be carried out for example at temperatures less than about 400° C., wherein an anneal is carried out subsequently to the PECVD process.

According to various embodiments, the chemical vapor deposition process that may be used to carry out step 210 of method 200 may include a hydrocarbon, e.g. as pre-cursor or source material in gaseous form. The hydrocarbon may be or may include an alkane, e.g. CH₄, an alkene, e.g. C₂H₄, an alkyne, e.g. C₂H₂, or an aromatic hydrocarbon (also referred to as arene), e.g. C₆H₆. According to various embodiments, other suitable carbon containing source materials may be used for the chemical vapor deposition process. Further, additional hydrogen may be added, e.g. controlled, during the CVD process, e.g. hydrogen in gas form (H₂).

According to various embodiments, hydrogenated amorphous carbon deposited via chemical vapor deposition (cf. step 210 of method 200) may include for example a molar percentage of hydrogen in the range from about 20% to about 60% and a molar percentage of sp³-hybridized carbon in the range from about 20% to about 65%.

According to various embodiments, the chemical vapor deposition process may be carried out at a temperature of less than about 500° C., wherein the layer may be annealed at a temperature greater than about 700° C. Plasma parameters for the CVD process may include an RF-frequency in the range from about 4 kHz to about 80 MHz, an RF-power in the range from about 10 W to about 10 kW, and a pressure in the range from about 0.1 mbar to about 100 mbar. A hydrogenated amorphous carbon layer deposited at a temperature of less than about 500° C. via CVD may have a specific electrical resistance greater than about 1 Ohm·m. Further, annealing the layer at a temperature greater than about 700° C. may reduce the specific electrical resistance of the layer to a value of less than about 200 μOhm·m. According to various embodiments, the hydrogenated amorphous carbon layer may be annealed at a temperature greater than about 850° C. so that the specific electrical resistance of the formed turbostratic graphite layer is less than about 100 μOhm·m. According to various embodiments, the hydrogenated amorphous carbon layer may be annealed at a temperature greater than about 950° C. so that the specific electrical resistance of the formed turbostratic graphite layer is less than about 80 μOhm·m. According to various embodiments, the hydrogenated amorphous carbon layer may be annealed at a temperature greater than about 1100° C. so that the specific electrical resistance of the formed turbostratic graphite layer is less than about 60 μOhm·m. According to various embodiments, the annealing temperature may be less than about 1500° C., i.e. less than the crystallization temperature of graphite.

According to various embodiments, the annealing may be carried out for an annealing duration of about 1 min to about 15 min, e.g. for 4 min. A longer annealing duration, e.g. 15 min or longer, may reduce the specific electrical resistance of the layer to a lower value than a shorter annealing duration, e.g. 1 min. According to various embodiments, the annealing duration may be at least 1 min.

If the turbostratic graphite is deposited by a thermal CVD process at a temperature greater than about 650° C. or greater than about 700° C.; and at pressures in the range from about 0.1 mbar to about atmospheric pressure, a further annealing process after the deposition process may not be necessary. Illustratively, the layer may be already annealed during deposition or, in other words, the layer may grow thermodynamically controlled.

Single crystalline graphite may have an anisotropic specific electrical resistance parallel to the lattice planes (i.e. parallel to the hexagonally arranged carbon sheets) and perpendicular to the lattice planes, also referred to as βₚₐᵣₐₗₗₑₗ and β_{perpendicular}. The anisotropy factor regarding the electrical resistivity (ρ_{perpendicular}/ρₚₐᵣₐₗₗₑₗ) of single crystalline graphite 102 may be greater than the anisotropy factor of turbostratic graphite 120, cf. FIG. 1. Illustratively, the disorder of turbostratic graphite may reduce to anisotropy compared to the lamellae structure of single crystalline graphite 102. According to various embodiments, turbostratic graphite may include nanocrystalline graphite cluster and therefore may have an anisotropy factor of the specific electrical resistance of less than about 1000, e.g. less than about 100 or less than about 10. According to various embodiments, the specific electrical resistance or the electrical conductivity of a layer may be measured in 4-point probe method (also referred to as four-terminal sensing or 4-wire sensing). Alternatively, a less accurate 2-point probe method may be used.

According to various embodiments, the annealing may include Rapid Thermal Processing (RTP) including heating rates of about 100 Kelvin per second, e.g. using a lamp heater of a flash lamp. A laser (e.g. a XeCl Excimer-Laser) may be used for locally annealing the layer. The annealing may be carried out in a chemically inert atmosphere, e.g. in absence of oxygen. A chemically inert atmosphere may include nitrogen, hydrogen and/or argon. Alternatively, the annealing may be carried out using a furnace or any other suitable annealing technique.

According to various embodiments, a hydrogen content of the layer including or essentially consisting of hydrogenated amorphous carbon may be reduced during the annealing. A hydrogenated amorphous carbon layer deposited via CVD at a temperature of less than about 500° C. may have a molar percentage of hydrogen greater than about 20%, wherein the molar percentage of hydrogen is reduced to less than about 10% or less than about 5% during annealing. However, since the annealing temperature may be less than about 2000° C. or less than about 1500° C., residual hydrogen may remain in the layer after annealing, e.g. with a molar percentage in the range from about 1% to about 10%, or with a molar percentage in the range from about 1% to about 5%.

According to various embodiments, the annealing of the hydrogenated amorphous carbon layer may be carried out only after deposition. According to various embodiments, the annealing of the hydrogenated amorphous carbon layer may be carried out during deposition. Further, the annealing of the hydrogenated amorphous carbon layer may be carried out during and after deposition.

According to various embodiments, the method 200 may further include: forming an electrode structure that electrically contacts the turbostratic graphite layer formed from the hydrogenated amorphous carbon layer. The electrode structure may be formed before or after the annealing of the hydrogenated amorphous carbon layer.

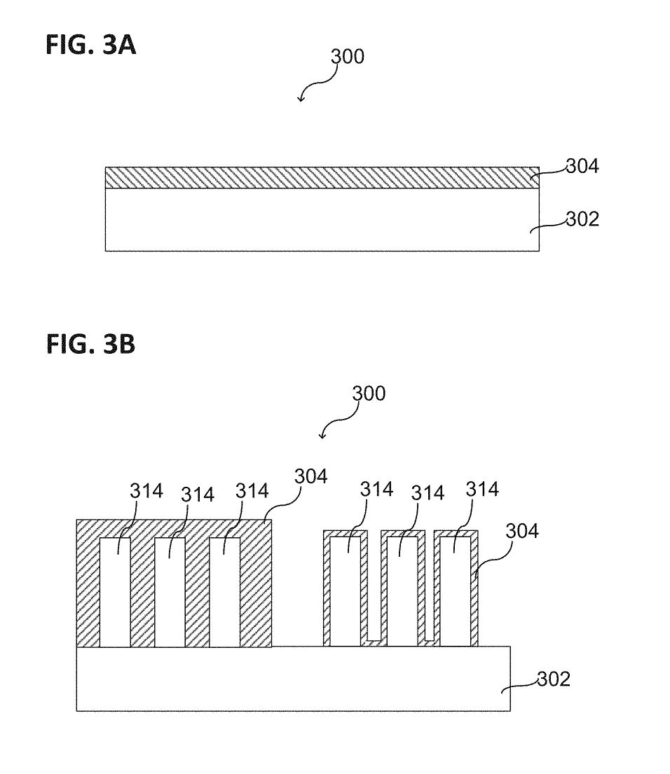

FIG. 3A illustrates a schematic cross sectional view of a carrier 302 during application of method 200 or after method 200 has been carried out. According to various embodiments, a layer 304 may be disposed over the carrier 302, wherein the layer 304 includes or essentially consists of turbostratic graphite. The layer 304 may be a sensor layer. According to various embodiments, the carrier 302 may be electrically insulating, so that electrical properties of the layer 304 can be evaluated accurately. According to various embodiments, in case the carrier 302 may be electrically conductive or semiconductive, an additional isolation structure may be disposed between the carrier 302 and the layer 304 electrically separating the layer 304 from the carrier 302. According to various embodiments, the carrier 302 may be a silicon wafer or any other type of suitable carrier.

According to various embodiments, the carrier 302 may include one or more structure elements 314 or one or more structure elements 314 may be disposed on the carrier 302, as illustrated in FIG. 3B in a schematic cross sectional view. The layer 304 including turbostratic graphite or essentially consisting of turbostratic graphite may cover (e.g. conformally cover, e.g. partially or completely cover) the structure elements 314 of the carrier 302. The structure elements 314 may include or essentially consist of the same material as the carrier 302. Alternatively, the structure elements 314 may be or may include any type of structure processed in semiconductor processing, as for example a transistor structure, a diode structure or any other type of integrated circuit structure. According to various embodiments, the carrier 302 may include a driver circuit and/or a measurement circuit electrically contacting the layer 304. Electronic properties of the turbostratic graphite layer 304 may be measured using the measurement circuit. Therefore, the turbostratic graphite layer 304 may be used as a sensor layer. Further, the sensor layer 304 may be heated up to a predefined temperature using the driver circuit, thereby an adsorbed material (e.g. gas, water, etc.) may be removed from the sensor layer 304.

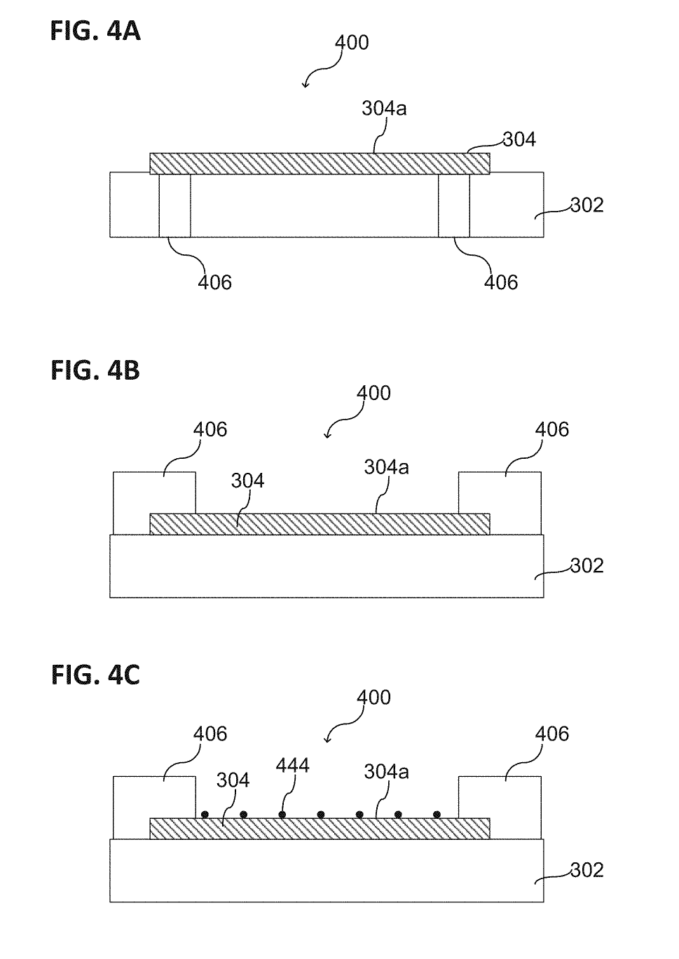

FIG. 4A and FIG. 4B respectively illustrate a sensor 400 in a schematic cross sectional view, according to various embodiments. The sensor 400 may include a carrier 302 and a sensor layer 304 disposed on the carrier 302, as described for example with reference to FIGS. 3A and 3B. The carrier 302 may include an electrode structure 406, e.g. at least two electrodes laterally spaced apart from each other. The electrode structure 406 may be formed in the carrier 302 (as exemplarily shown in FIG. 4A) and/or the electrode structure 406 may be formed over the carrier 302 and at least partially over the sensor layer 304 (as exemplarily shown in FIG. 4B). Alternatively, the electrode structure 406 may be formed over the carrier 302 and may laterally contact the sensor layer 304. The sensor layer 304 may be in contact (e.g. in direct physical and/or electrical contact) with the electrode structure 406 and may include or essentially consist of turbostratic graphite. At least a part of a surface 304a of the sensor layer 304 may be free of any solid material so that a gas, humidity, bio-molecules, etc. may have direct access to the sensor layer 304. Therefore, the electronic properties of the sensor layer 304 may be influenced by presence of a gas, humidity, bio-molecules, and the like.

According to various embodiments, the electrode structure 406 may include at least two electrodes contacting the sensor layer 304 so that a two-point probe method can be carried out to resistively measure the electrical properties of the sensor layer 304. According to various embodiments, the electrode structure 406 may include at least four electrodes contacting the sensor layer 304 so that a 4-point probe method can be carried out to resistively measure the electrical properties of the sensor layer 304. Alternatively, the electrode structure 406 may be configured to capacitively measure the electrical properties of the sensor layer 304.

According to various embodiments, the turbostratic graphite of the sensor layer 304 is polycrystalline, as already described herein. The turbostratic graphite of the sensor layer 304 may have an average size of the crystallites of less than about 100 nm. In other words, the average size of the crystallites may be in the nanometer range, e.g. in the range from about 1 nm to about 20 nm.

According to various embodiments, the electrode structure 406 may be part of a measurement circuit or may be electrically connected to a measurement circuit, wherein the measurement circuit may be configured to determine an electrical property (e.g. the resistivity and/or impedance) of the sensor layer 304. Further, the measurement circuit may be configure to provide an (e.g. analog) output signal representing a concentration of a gas, a humidity, or a concentration of bio-molecules sensed by the sensor layer 304.

According to various embodiments, the electrode structure 406 may be part of a heat circuit (also referred to as driver circuit) or may be electrically connected to a heat circuit, wherein the heat circuit may be configured to heat the sensor layer via an electrical current to a predefined temperature.

As used herein, a “circuit” may be understood as any kind of analog or digital implementing entity, which may be special purpose circuitry or a processor executing software stored in a memory, firmware, hardware, or any combination thereof.

FIG. 4C illustrates a sensor 400 in a schematic cross sectional view, according to various embodiments. The sensor 400 may include a surface coating 444 at least partially covering the sensor layer 304. The surface coating 444 may be in direct physical contact with a surface 304a of the sensor layer 304. According to various embodiments, the surface coating 444 may include a patterned coating layer or a plurality of particles, e.g. nanoparticles.

According to various embodiments, the sensor 400 may be configured as gas sensor. The sensor layer 304 may be sensitive to specific gases, e.g. NH₃ or N₂O, without any surface coating. However, the surface coating 444 may be configured to adjust or improve the sensitivity of the sensor layer 304 for a target gas, e.g. carbon monoxide. Therefore, the surface coating 444 may include a plurality of nanoparticles. The nanoparticles may include or may essentially consist of a metal or a metal oxide. The nanoparticles may include or may essentially consist of copper and/or nickel.

According to various embodiments, the sensor 400 may be configured as humidity sensor. The sensor layer 304 may be sensitive to water vapor without any surface coating. However, the surface coating 444 may be configured to improve the sensitivity of the sensor layer 304 for water vapor.

According to various embodiments, the sensor 400 may be configured as bio-molecule sensor. According to various embodiments, the surface coating 444 may be configured to adjust the sensitivity of the sensor layer 304 for bio-molecules, e.g. via capture bio-molecules immobilized on the surface 304a of the sensor layer 304 and configured to hybridize bio-molecules to be detected.

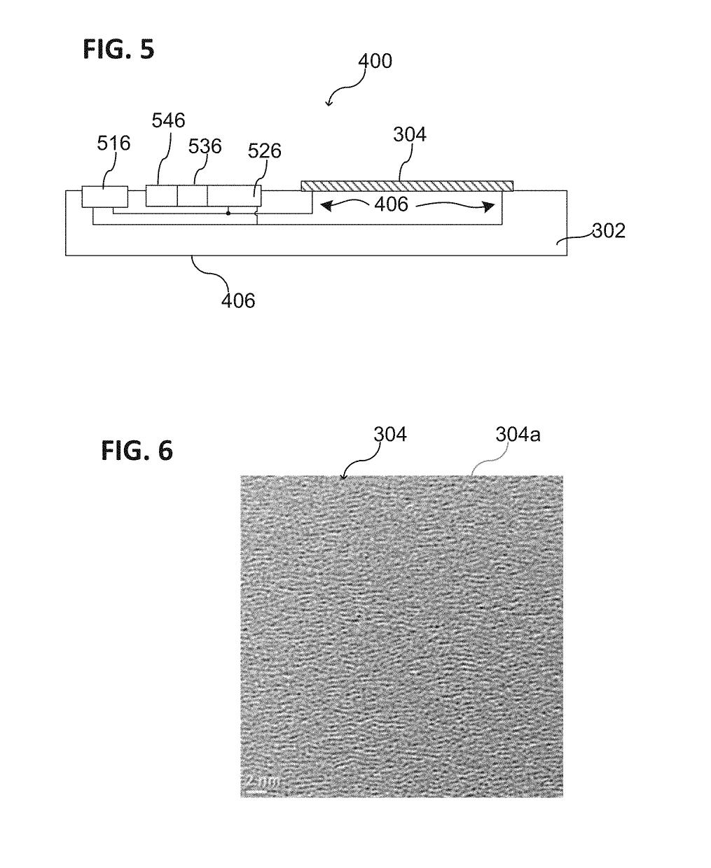

FIG. 5 illustrates a sensor 400 in a schematic cross sectional view, according to various embodiments. The sensor 400 may include a carrier 302 with an electrode structure 406 and a sensor layer 304 in contact with the electrode structure 406, wherein the sensor layer 304 may include or essentially consist of turbostratic graphite. According to various embodiments, the sensor layer 304 may be configured as described before.

According to various embodiments, the sensor 400 may include a heat circuit 516 coupled to the electrode structure 406 and configured to heat the sensor layer 304 via an electrical current. Further, the sensor 400 may include a measurement circuit 526 coupled to the electrode structure 406 and configured to determine at least one electrical property (e.g. the electrical resistivity or the impedance) of the sensor layer 304. An impedance measurement may include measuring the ohmic impedance and the phase shift representing the capacitive impedance and the inductive impedance. A resistivity measurement may include measuring the ohmic resistivity or the ohmic resistance.

According to various embodiments, an analog-digital converter 536 may be connected to the measurement circuit 526 and configured to convert an analog measurement signal from the sensor layer 304 (e.g. based on the electrical resistance) to a digital measurement signal. Further, a signal processor 546 may be connected to the analog-digital converter and configured to provide an output signal based on the digital measurement signal, the output signal representing a concentration of a gas sensed by the sensor layer 304.

The signal processor 546 and the analog-digital converter 536 may be provided in semiconductor technology over and/or in the carrier 302. Therefore, the carrier may include or may essentially consist of semiconductor material, e.g. silicon. The sensor layer 304 may be electrically isolated from a semiconductor carrier 302 or from semiconductor material of the carrier 302, e.g. via at least one electrically insulating layer. Further, the electrode structure 406 may be electrically isolated from a semiconductor carrier 302 or from semiconductor material of the carrier 302. The electrode structure 406 may be formed as metallization layer including a wiring structure embedded in (e.g. low-k) dielectric material.

According to various embodiments, the sensor 400 may be operated alternatingly including a measurement step and subsequently a heating step to prepare the sensor layer 304 for the following measurement step.

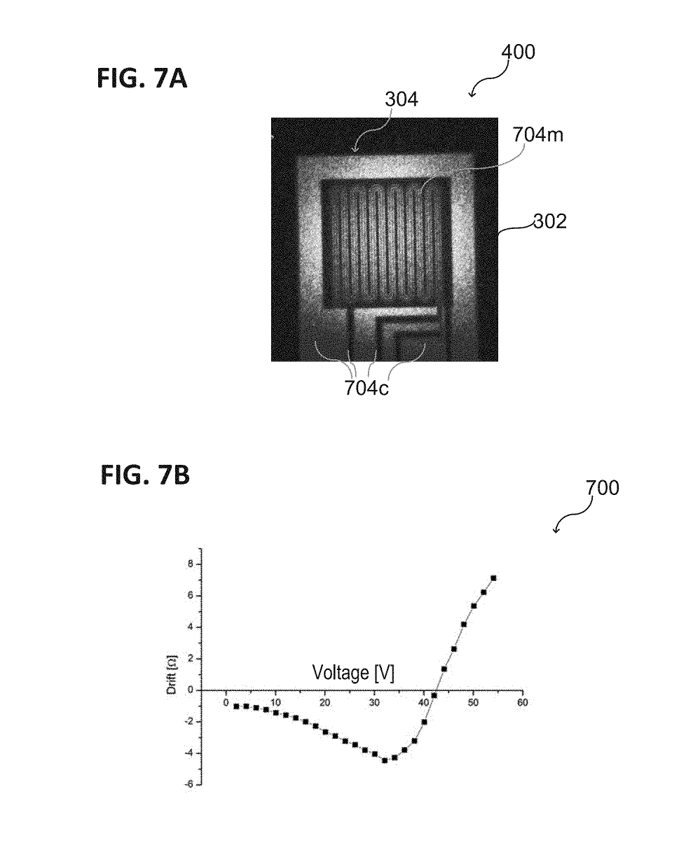

FIG. 6 illustrates a top view of a sensor layer 304 of a sensor 400 by electron microscopy, according to various embodiments. The sensor layer 304 may be disordered or in other words may not have a single crystalline graphite structure. The sensor layer 304 may include polycrystalline graphite with an average crystallite size in the nanometer range. The surface 304a of the sensor layer 304 may have a surface roughness greater than about 0.3 nm, e.g. in the range from about 1 nm to about 3 nm. The surface roughness may be measured via scanning force microscopy, wherein the values represent the RMS (Root mean square) surface roughness for example based on an analyzed surface area of 1 μm².

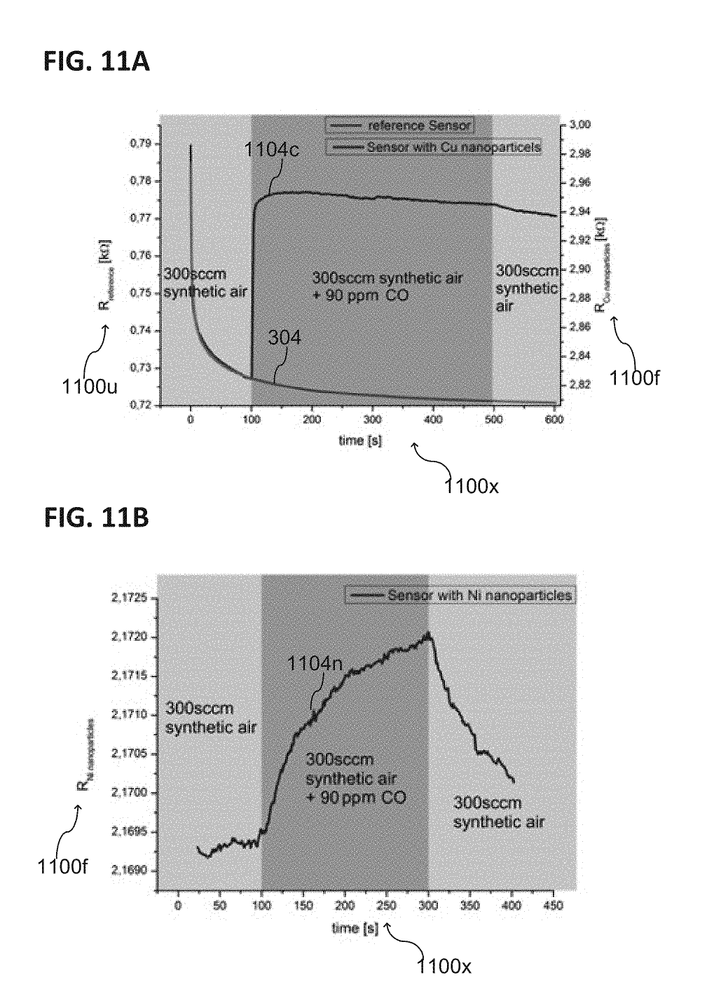

It was found that the turbostratic graphite is not only sensitive towards humidity, but also towards other gases like ammonia or nitrous oxide. Further, the turbostratic graphite can be functionalized by metal particles (e.g. nanoparticles) to modify the sensitivity and thus the selectivity to several gases other than water vapor, see for example FIG. 11A and FIG. 11B. According to various embodiments, hydrogenated amorphous carbon may be transformed to turbostratic graphite during a high-temperature treatment (also referred to as annealing) and the annealed hydrogenated amorphous carbon is used as sensitive layer. Hydrogenated amorphous carbon can be produced easily by, for example, PECVD with carbon containing gases like methane or ethane as pre-cursor. Annealing of the hydrogenated amorphous carbon layers leads to outgassing of the bound hydrogen and to crystallization of hydrogenated carbon. The annealed hydrogenated amorphous carbon is graphite-like with small crystallite sizes in the nanometer range and electrically conductive.

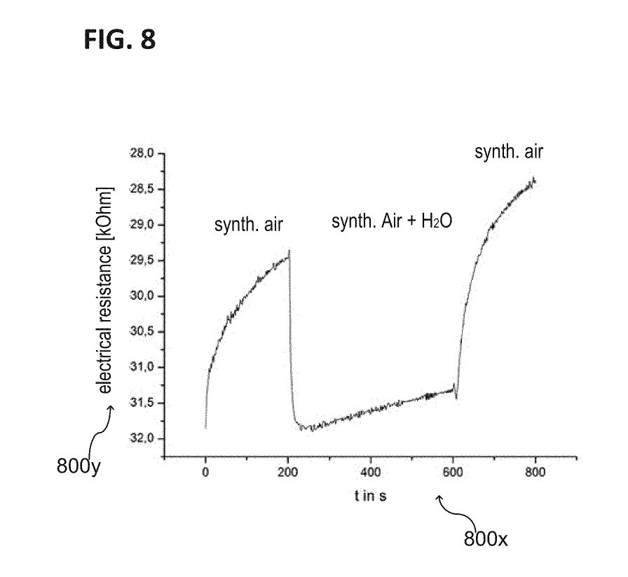

FIG. 7A illustrates a top view of a sensor 400, according to various embodiments. The sensor layer 304 may be patterned, e.g. via a lithographic mask process and an etch process, e.g. via dry etching, to partially remove the material of the sensor layer 304. The patterned sensor layer 304 may include a meander shaped line structure 704m that is, for example, the sensing part of the patterned sensor layer 304 and contact areas 704c for electrically contacting the meander shaped line structure 704m.

According to various embodiments, the sensor layer 304 itself may be used as heating element by providing a suitably high heating current (i.e., a current that causes heat to be produced to achieve a desorption of gas or humidity from the sensor layer 304) through the sensor layer 304. FIG. 7A shows a thermographic image of a self-heated patterned sensor layer 304, according to various embodiments. The sensor layer 304 may have a thickness greater than about 1 nm, e.g. a thickness in the range from about 2 nm to about 100 nm, e.g. in the range from about 2 nm to about 40 nm. FIG. 7B shows a measurement of the resistance drift for an increasing voltage applied on the patterned sensor layer 304. From 0 V to about 30 V the resistance drift is increasing due to thermal adaption of the environment. From 30 V to higher voltages water starts to desorb from the sensor. Thus, the sensor layer 304 including turbostratic graphite can be used as heater-structure.

Alternatively, an additional heating element may be provided spaced apart from the sensor layer 304. The heating element may be provided in and/or over the carrier 302 adjacent to the sensor layer 304 to indirectly heat the sensor layer 304. In this case, a driver circuit (also referred to as heat circuit) may be connected to the heating element, wherein the driver circuit is configured to operate the heating element, e.g. by providing a suitably high heating-current through the heating element.

According to various embodiments, the heating of the sensor layer 304 may be necessary or helpful for sensing certain gases or water, wherein the heating can be indirectly done (e.g. by an external hotplate) or directly by providing a current through the sensor layer 304 itself, as described herein. The direct heating using the sensor layer 304 itself as a heater may be compared to many commercially available products that need separate heater structures. According to various embodiments, the use of the sensor layer 304 as heating structure may reduce manufacturing costs and power consumption of the sensor 400.

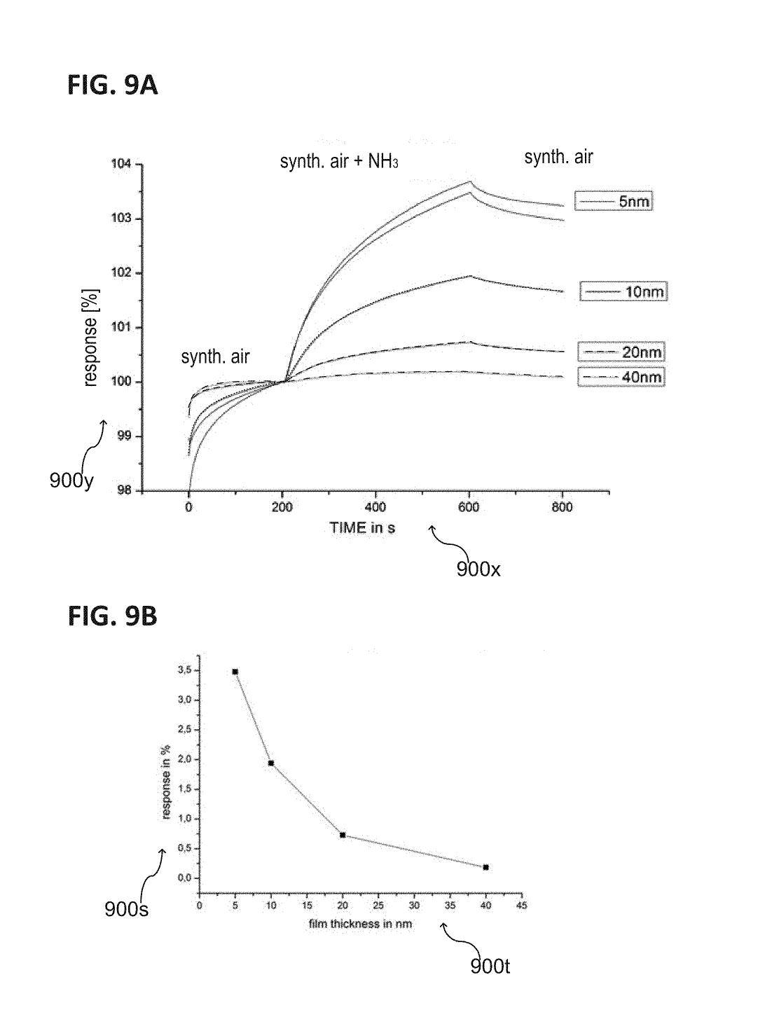

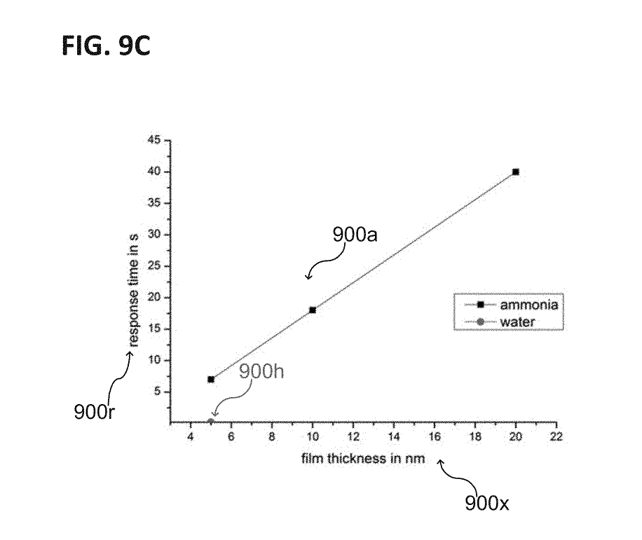

FIG. 8 shows a time curve 800x of the electrical resistance 800y of a 5 nm thick sensor layer 304, while the sensor layer 304 is exposed to synthetic air (that is free of water), subsequently to a mixture of synthetic air and water vapor (simulating a humidity exposure), and subsequently to synthetic air. As can be seen from the resistivity change upon humidity exposure, turbostratic graphite changes its electrical resistance upon adsorption of gases (e.g. humidity, ammonia, nitrous oxide, etc.). The sensitivity towards specific gases can be increased by functionalization of the material with metal and metal oxide coatings, e.g. nanoparticles, see for example FIG. 12. The resistance change with humidity exposure is rather high (e.g. up to 60%), leading to a highly sensitive sensor based on turbostratic graphite (see FIG. 9A and FIG. 9B). According to various embodiments, the resistance change may depend on the average crystallite size and the residual hydrogen amount of the sensor layer 304.

FIG. 9A to FIG. 9C respectively illustrate sensor characteristics (e.g. the response, the sensitivity, and the response time) of a turbostratic graphite sensor layer 304, according to various embodiments. The sensor characteristics can be tuned by adjusting the thickness of the sensor layer 304. Alternatively, the sensor characteristics may be tuned by other measures, like adapting the used source gas for the used CVD process, adapting the anneal parameters, and the like.

FIG. 9A shows a response 900y of the sensor layer 304 over time 900x when being exposed to ammonia (NH₃). The sensor layer 304 is thereby exposed to synthetic air until time point 200 s, to synthetic air and ammonia in a period of time from 200 s to 600 s, and to synthetic air again from time point 600 s. The response 900y represents the normalized change of the electrical resistance of the sensor layer 304. As can be seen from FIG. 9A, the response 900y increases with decreasing thickness of the sensor layer 304. The correlation between the maximal response 900s (also referred to as sensitivity 900s) and the thickness 900t of the turbostratic graphite layer 304 is illustrated in FIG. 9B. The response is measured for an exposure of 50 ppm ammonia in synthetic air.

According to various embodiments, the sensor layer 304 may have a thickness of less than about 40 nm. The sensor layer 304 may have a thickness greater than about 1 nm or 2 nm. According to various embodiments, the thickness may be selected so that the sensor layer 304 is dense and allows a lateral current flow to measure the electronic properties resistively. Alternatively, a capacitive measurement may be applied.

FIG. 9C illustrates a correlation between the response time 900r and the thickness of the sensor layer 304, according to various embodiments. The response time corresponds to the time needed for a relative change of the electrical resistance of 0.2%. The response time is measured for the exposure of both, water 900h and ammonia 900a.

The sensor 400 may essentially consist of turbostratic graphite layer that may be electrically contacted from top and/or bottom or from the side. Two or four electrodes may be used to electrically contact the sensor layer 304. According to various embodiments, a gold meander electrode may be used to electrically contact the sensor layer 304. However, the meander electrode may include any other suitable material, e.g. a metal or polycrystalline silicon. The resistance change upon an exposure to humidity or to a desired gas may be very fast, as shown for example in FIG. 9C, and can be easily detected by a 2-point or 4-point measurement.

According to various embodiments, the concept of the gas or humidity sensor 400 described herein may allow good reproducibility during manufacture due to a low production complexity. Further, the gas or humidity sensor 400 may have a fast and high response and a high chemical stability against aggressive environmental gases. The manufacturing may be possible with standard semiconductor process tools only. Further, the scaling-down potential is high due to the applied layering technique.

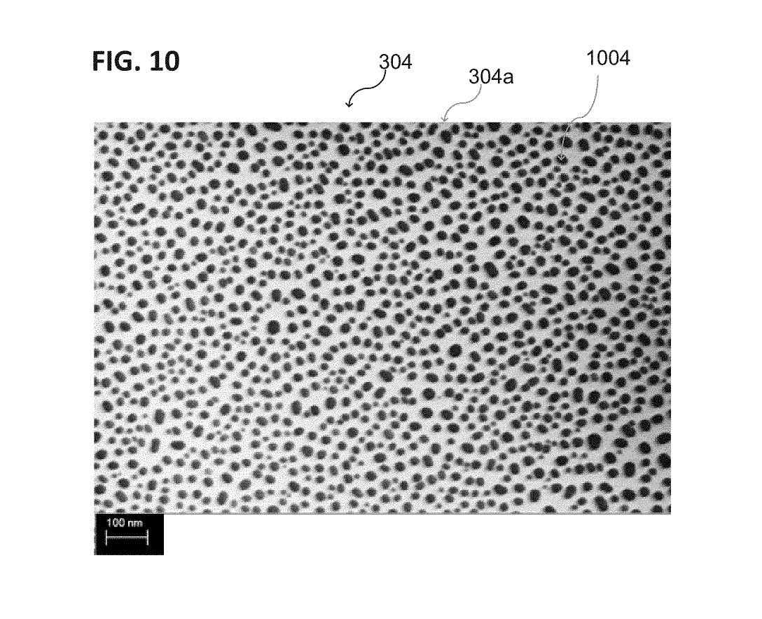

FIG. 10 illustrates a top view of a sensor layer 304 of a gas or humidity sensor 400 by electron microscopy, according to various embodiments. The sensor layer 304 may be partially covered with a functionalizing material 1004 (the functionalizing material 1004 corresponds to the bright dots of the electron microscopy image). The functionalizing material 1004 may be formed by electroplating, evaporation, sputtering, and the like. The functionalizing material 1004 may be or may include a metal and/or a metal oxide, e.g. in form of nanoparticles, as illustrated in FIG. 10. According to various embodiments, the functionalizing material 1004 may change the response of the sensor layer 304 to specific gases, see FIG. 11A and FIG. 11B.

FIG. 11A illustrates a time curve 1100x of the electrical resistance 1100f (left scale) of a functionalized sensor layer 1104c during an exposure to carbon monoxide. For comparison, the electrical resistance 1100u (left scale) of a non-functionalized sensor layer 304 during an exposure to carbon monoxide is shown as reference. The functionalized sensor layer's response 1104c to the carbon monoxide exposure at time point 100 s shows an introduction of sensitivity of the functionalized sensor layer to carbon monoxide gas. This carbon monoxide sensitivity is caused by a functionalization of the sensor layer. For introduction of a carbon monoxide sensitivity, the functionalizing material may include copper nanoparticles, as illustrated in FIG. 11A.

FIG. 11B illustrates the electrical resistance 1100f over time 1100x for a functionalized sensor layer 1104n during an exposure of the functionalized sensor layer to carbon monoxide. The sensor response to carbon monoxide exposure at time point 100 s shows an introduction of carbon monoxide gas sensitivity of the functionalized layer 1104n by functionalization. For sensing carbon monoxide, the functionalizing material may include nickel nanoparticles, as illustrated in FIG. 11B. The gas sensor 400 including the sensor layer 304 functionalized with nickel nanoparticles (shown in FIG. 11B) has a better recovery performance than the gas sensor 400 including the sensor layer functionalized with copper nanoparticles as illustrated in FIG. 11A. The gas sensor 400 including the sensor layer functionalized with copper nanoparticles shows a faster initial response to carbon monoxide exposure than the gas sensor 400 including the sensor layer functionalized with nickel nanoparticles.

Various embodiments relate to the use of turbostratic graphite that is obtained from annealed PECVD hydrogenated amorphous carbon (a-C:H) for humidity or gas sensors. This material is cheap, fast (e.g. compared to a metal oxide (MOX) gas sensor or an electrochemical gas sensor, which may use higher film thicknesses (and are based on diffusion)), sensitive towards target gases, and resistant against aggressive environmental gases. Further, turbostratic graphite can be produced by standard semiconductor equipment. The sensitivity of a turbostratic graphite layer towards specific gases can be improved by surface functionalization, e.g. by metal or metal oxide nanoparticles or other surface coatings. Turbostratic graphite can be formed homogenously over a surface of a carrier by deposition and annealing, as described herein. An a-C:H layer may generally be deposited by plasma-enhanced CVD at temperatures less than about 400° C. or less than about 500° C. The subsequent anneal, according to various embodiments, at a temperature of about 700° C. or greater than 700° C. to form turbostratic graphite can be done by furnace, RTP or laser anneal. Using a laser anneal, the device temperature (i.e. the temperature of the carrier 302 below the a-C:H layer) can be kept at lower temperatures due to the fast heat input at the surface of the a-C:H layer. Alternatively, a direct thermal CVD process may be applied thereby forming nanocrystalline turbostratic graphite using a hydrocarbon precursor (e.g. CH₄, C₂H₂) at deposition temperatures of about 700° C. or greater than about 700° C.

In analogy to a surface coating that provides sensitivity towards specific gases, a surface coating may be used to provide sensitivity towards bio-molecules. In this case, the surface coating may be configured to capture specific bio-molecules. The captured bio-molecules may change the resistance or the capacitance of the sensor layer 304, which can be detected by a measurement circuit, as described herein. Therefore, the surface coating may include at least one capture molecule immobilized on the sensor layer 304 and configured to hybridize bio-molecules to be detected. Illustratively, the bio-molecules to be sensed may be bound to the surface 304a of the sensor layer 304 by covalent bonding and/or electrostatic, hydrophobic and Van-der-Waals interaction caused by the capture molecule immobilized at the surface 304a of the sensor layer 304. Alternatively, the bio-molecules to be sensed may be bound directly to a surface of the sensor layer 304.

Example 1 is a sensor for sensing a fluid, e.g. a gas, humidity, bio-molecules, and the like. The sensor may include: a carrier including an electrode structure; and a sensor layer in contact with the electrode structure, wherein the sensor layer includes or essentially consists of turbostratic graphite. Alternatively, the sensor may include: a carrier; an electrode structure disposed over and/or in the carrier; and a sensor layer in contact with the electrode structure, wherein the sensor layer includes or essentially consists of turbostratic graphite. Alternatively, the sensor may include: a carrier including an electrode structure; and a sensor layer in contact with the electrode structure, wherein the sensor layer includes or essentially consists of disordered graphite. Alternatively, the sensor may include: a carrier including an electrode structure; and a sensor layer in contact with the electrode structure, wherein the sensor layer includes or essentially consists of rotationally disordered graphite.

In Example 2, the subject matter of Example 1 can optionally include that the sensor layer is in direct electrical and/or direct physical contact with the electrode structure.

In Example 3, the subject matter of Example 1 or 2 can optionally include that the sensor is a gas sensor or is configured as gas sensor.

In Example 4, the subject matter of Example 3 can optionally include that the gas sensor further includes: a surface coating at least partially covering the sensor layer, wherein the surface coating is configured to adjust the sensitivity of the sensor layer for a target gas.

In Example 5, the subject matter of Example 1 or 2 can optionally include that the sensor is a humidity sensor.

In Example 6, the subject matter of Example 5 can optionally include that the humidity sensor further includes: a surface coating at least partially covering the sensor layer, wherein the surface coating is configured to adjust the sensitivity of the sensor layer for humidity.

In Example 7, the subject matter of Example 1 or 2 can optionally include that the sensor is a bio-molecule sensor.

In Example 8, the subject matter of Example 7 can optionally include that the bio-molecule sensor further includes: a surface coating at least partially covering the sensor layer, wherein the surface coating is configured to adjust the sensitivity of the sensor layer for bio-molecules.

In Example 9, the subject matter of Example 8 can optionally include that the surface coating may be configured to capture bio-molecules.

In Example 10, the subject matter of Example 8 or 9 can optionally include that the surface coating includes at least one capture molecule immobilized on the sensor layer and configured to hybridize bio-molecules to be detected.

In Example 11, the subject matter of any one of Examples 1 to 10 can optionally include that the surface coating may include a plurality of nanoparticles.

In Example 12, the subject matter of Example 11 can optionally include that the nanoparticles include or essentially consist of a metal or a metal oxide.

In Example 13, the subject matter of Example 11 or 12 can optionally include that the nanoparticles include or essentially consist of copper and/or nickel.

Alternatively, the subject matter of any one of Examples 1 to 10 can optionally include that the surface coating may include a patterned layer. Further, the surface coating may include or essentially consist of a metal or a metal oxide. Further, the surface coating may include or essentially consist copper and/or nickel

In Example 14, the subject matter of any one of Examples 1 to 13 can optionally include that the sensor layer may have a thickness of less than about 40 nm, e.g. less than about 30 nm or less than about 20 nm.

In Example 15, the subject matter of any one of Examples 1 to 14 can optionally include that the sensor layer has a thickness greater than about 2 nm, e.g. greater than 3 nm or greater than 4 nm.

In Example 16, the subject matter of any one of Examples 1 to 15 can optionally include that the carrier may be a dielectric carrier or may include dielectric material.

In Example 17, the subject matter of any one of Examples 1 to 16 can optionally include that the carrier further includes silicon or any other semiconductor material.

In Example 18, the subject matter of any one of Examples 1 to 17 can optionally include that the sensor layer is electrically insulated from the carrier, e.g. by at least one electrically insulating layer.

In Example 19, the subject matter of any one of Examples 1 to 18 can optionally include that the sensor further includes: a measurement circuit connected to the electrode structure and configured to determine an electrical property of the sensor layer.

In Example 20, the subject matter of Example 19 can optionally include that the electrical property includes or is a resistivity of the sensor layer or an impedance of the sensor layer.

In Example 21, the subject matter of any one of Examples 1 to 20 can optionally include that the sensor further includes: an analog-digital converter connected to the measurement circuit and configured to convert an analog measurement signal generated by the sensor layer to a digital measurement signal.

In Example 22, the subject matter of any one of Examples 1 to 21 can optionally include that the sensor further includes: a signal processor connected to the analog-digital converter and configured to provide an output signal based on the digital measurement signal.

In Example 23, the subject matter of any one of Examples 1 to 22 can optionally include that the sensor is a gas sensor and that the output signal represents a concentration of a gas sensed by the sensor layer.

In Example 24, the subject matter of any one of Examples 1 to 22 can optionally include that the sensor is a humidity sensor and that the output signal represents an absolute humidity and/or a relative humidity sensed by the sensor layer. The absolute humidity is the ratio of the mass of the water vapor to the volume of the mixture including dry air and the water vapor. The relative humidity is the ratio of the partial pressure of water vapor to the equilibrium vapor pressure of water at a given temperature.

In Example 25, the subject matter of any one of Examples 1 to 22 can optionally include that the sensor is a bio sensor or bio-molecule sensor and that the output signal represents a concentration of bio-molecules sensed by the sensor layer. According to various embodiments, the sensor may be calibrated by one or more reference measurements for measuring a concentration of bio-molecules. The reference measurements may take the adsorption/desorption equilibrium in the analyzed media into account. Alternatively, the output signal may represent a number of bio-molecules sensed by the sensor layer. According to various embodiments, a bio-molecule may be based on a hydrocarbon.

In Example 26, the subject matter of any one of Examples 1 to 25 can optionally include that sensing a concentration of gas, humidity, or bio-molecules may include detecting the absence or presence of the respective gas or bio-molecules.

In Example 27, the subject matter of any one of Examples 1 to 26 can optionally include that the carrier is made of silicon or includes silicon and that the measurement circuit, the analog-digital converter, and/or the signal processor may be formed in CMOS technology in and/or over the carrier.

In Example 28, the subject matter of any one of Examples 1 to 27 can optionally include that the sensor layer is formed directly on (any suitable) electrically insulating material.

In Example 29, the subject matter of any one of Examples 1 to 28 can optionally include that the sensor further includes: a driver circuit connected to the electrode structure and configured to heat the sensor layer by providing a heating current through the sensor layer.

In Example 30, the subject matter of Example 29 can optionally include that the carrier is made of silicon or includes silicon and that the driver circuit for heating the sensor layer is formed in CMOS technology in and/or over the carrier.

In Example 31, the subject matter of any one of Examples 1 to 28 can optionally include that the sensor further includes: a heating element configured to heat the sensor layer; and a driver circuit connected to the heating element, wherein the driver circuit is configured to operate the heating element.

Example 32 is a method for forming a sensor layer. The method may include: depositing a hydrogenated amorphous carbon layer over a carrier; and annealing the hydrogenated amorphous carbon layer to form turbostratic graphite from the hydrogenated amorphous carbon. Alternatively, the method for forming a sensor layer may include: depositing a layer over a carrier by chemical vapor deposition of a hydrocarbon precursor, the layer including hydrogenated amorphous carbon; and annealing the layer to form turbostratic graphite from the hydrogenated amorphous carbon. Further, annealing the layer may include to transform the hydrogenated amorphous carbon into turbostratic graphite.

In Example 33, the subject matter of Example 32 can optionally include that the chemical vapor deposition process is a plasma-enhanced chemical vapor deposition process.

In Example 34, the subject matter of Example 32 or 33 can optionally include that the chemical vapor deposition process is carried out at a temperature of less than about 500° C.

In Example 35, the subject matter of any one of Examples 32 to 34 can optionally include that annealing the layer may be carried out at a temperature greater than about 700° C.

In Example 36, the subject matter of any one of Examples 32 to 35 can optionally include that annealing the layer may be carried out at a temperature of less than about 2000° C., e.g. less than about 1500° C.

In Example 37, the subject matter of any one of Examples 32 to 36 can optionally include that annealing the layer to form turbostratic graphite from the hydrogenated amorphous carbon further reduces a hydrogen content of the layer.

In Example 38, the subject matter of any one of Examples 32 to 37 can optionally include that annealing the layer to form turbostratic graphite from the hydrogenated amorphous carbon further reduces an electrical resistivity of the layer. The electrical resistivity may be the specific electrical resistivity of the layer.

In Example 39, the subject matter of any one of Examples 32 to 38 can optionally include that annealing the layer is carried out after depositing the layer.

In Example 40, the subject matter of any one of Examples 32 to 39 can optionally include that annealing the layer is carried out during depositing the layer.

In Example 41, the subject matter of any one of Examples 32 to 40 can optionally further include: forming an electrode structure, the electrode structure electrically and/or physically contacting the layer.

Example 42 is a method for operating a fluid sensor. The method may include: removing adsorbed material from a sensor layer of the fluid sensor by heating the sensor layer by an electrical current driven through the sensor layer (e.g. via a driver circuit coupled to the sensor layer); and, subsequently, applying a fluid directly to the sensor layer and measuring a variation of an electronic property of the sensor layer (e.g. via a measurement circuit coupled to the sensor layer). Further, the sensor layer includes or essentially consists of turbostratic graphite.

In Example 43, the subject matter of Example 42 can optionally further include: providing a signal that represents a concentration or substance amount of a constituent of the fluid based on the variation of the electronic property of the sensor layer. The variation of the electronic property of the sensor may be determined by comparison of the electronic property of the sensor with and without the adsorbed analyte.

In Example 44, the subject matter of any one of Examples 1 to 43 can optionally include that the turbostratic graphite includes less than about 10 mol-% of hydrogen.

In Example 45, the subject matter of any one of Examples 1 to 44 can optionally include that turbostratic graphite includes more than 1 mol-% of hydrogen.

In Example 46, the subject matter of any one of Examples 1 to 45 can optionally include that turbostratic graphite includes more than about 95 mol-% of sp²-hybridized carbon.

In Example 47, the subject matter of any one of Examples 1 to 46 can optionally include that turbostratic graphite is electrically conductive.

In Example 48, the subject matter of Example 47 can optionally include that a resistivity of the turbostratic graphite is less than about 500 μOhm·m.

In Example 49, the subject matter of any one of Examples 1 to 48 can optionally include that an exposed surface of the sensor layer has an RMS surface roughness greater than about 0.3 nm.

In Example 50, the subject matter of any one of Examples 1 to 49 can optionally include that the turbostratic graphite is polycrystalline.

In Example 51, the subject matter of any one of Examples 1 to 50 can optionally include that turbostratic graphite is polycrystalline with an average size of the crystallites of less than about 100 nm.

In a further Example, the subject matter of Examples 11 can optionally include that the target gas is at least one of CO₂, CO, VOC, NO₂, and H₂ and wherein the surface coating (1004) comprises at least one surface coating of the following group of surface coatings: a metal nanoparticle or a metal layer; a metal chalcogenide nanoparticle or a metal chalcogenide layer; and organic ligand groups (covalently or non-covalently bound to the surface comprising an organic molecule with functional groups like e.g. amines, thiols, sulfoxides, alcohol, cabonyl and carboxylic groups; by way of example, functional groups having heteroatoms may be provided such as e.g. N, O, S, P, B, Si, or a halgen. The VOC target gas may include one or more volatile organic compounds.

According to various embodiments, the thickness and/or the crystallite size and/or the hydrogen content of turbostratic graphite in the sensor layer may be adapted to thereby influence (e.g. to increase) a sensitivity of the sensor layer towards humidity, gases and/or biomolecules.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A gas sensor, comprising:
a carrier comprising an electrode structure; and
a sensor layer in contact with the electrode structure, wherein the sensor layer comprises turbostratic graphite.

2. The gas sensor of claim 1,
wherein the turbostratic graphite comprises less than about 10 molar percent of hydrogen.

3. The gas sensor of claim 1,
wherein the turbostratic graphite comprises more than about 95 molar percent of sp²-hybridized carbon.

4. The gas sensor of claim 1,
wherein a resistivity of the turbostratic graphite is less than about 500 μOhm m.

5. The gas sensor of claim 1,
wherein the turbostratic graphite is polycrystalline.

6. The gas sensor of claim 1, further comprising:
a surface coating at least partially covering the sensor layer, wherein the surface coating is configured to adjust a sensitivity of the sensor layer for a target gas.

7. The gas sensor of claim 6,
wherein the surface coating comprises a plurality of nanoparticles.

8. The gas sensor of claim 7,
wherein the nanoparticles comprise a metal or a metal oxide.

9. The gas sensor of claim 6,
wherein the target gas is carbon monoxide and wherein the surface coating comprises at least one metal of the following group of metals: copper and nickel.

10. The gas sensor of claim 6,
wherein the target gas is at least one of CO₂, CO, VOC, NO₂, and H₂ and wherein the surface coating comprises at least one of the following group:
a metal nanoparticle or layer;
a metal chalcogenide nanoparticle or layer; and
organic ligand groups.

11. The gas sensor of claim 1,
wherein the sensor layer has a thickness of less than about 100 nm.

12. The gas sensor of claim 1,
wherein the sensor layer has a thickness greater than about 2 nm.

13. The gas sensor of claim 1,
wherein the carrier is a dielectric carrier.

14. The gas sensor of claim 1, further comprising:
a measurement-circuit connected to the electrode structure and configured to determine an electrical property of the sensor layer.

15. The gas sensor of claim 14, further comprising:
an analog-digital converter connected to the measurement-circuit and configured to convert an analog measurement signal obtained from the sensor layer to a digital measurement signal.

16. The gas sensor of claim 15, further comprising:
a signal processor connected to the analog-digital converter and configured to provide an output-signal based on the digital measurement signal, the output signal representing a concentration of a gas sensed by the sensor layer.

17. The gas sensor of claim 1, further comprising:
a driver circuit connected to the electrode structure and configured to heat the sensor layer by providing a heating current through the sensor layer.

18. The gas sensor of claim 1, further comprising:
a heating element to heat the sensor layer and a driver circuit connected to the heating element, wherein the driver circuit is configured to operate the heating element.

19. A humidity sensor comprising:
a carrier comprising an electrode structure; and
a sensor layer in contact with the electrode structure, wherein the sensor layer comprises or essentially consists of turbostratic graphite.

20. The humidity sensor of claim 19,
wherein the turbostratic graphite comprises less than about 10 molar percent of hydrogen.

21. The humidity sensor of claim 19,
wherein the turbostratic graphite comprises more than about 95 molar percent of sp²-hybridized carbon.

22. The humidity sensor of claim 19,
wherein a resistivity of the turbostratic graphite is less than about 500 μOhm m.

23. The humidity sensor of claim 19,
wherein the turbostratic graphite is polycrystalline.

24. The humidity sensor of claim 19, further comprising:
a measurement-circuit connected to the electrode structure and configured to determine an electrical property of the sensor layer and to provide an output signal representing a humidity of a gas sensed by the sensor layer.

25. A method for forming a sensor layer, the method comprising:
depositing a layer over a carrier by chemical vapor deposition of a hydrocarbon precursor, the layer comprising hydrogenated amorphous carbon; and
annealing the layer to transform the hydrogenated amorphous carbon into turbostratic graphite.

26. The method of claim 25,
wherein the turbostratic graphite comprises less than about 10 molar percent of hydrogen.

27. The method of claim 25,
wherein the turbostratic graphite comprises more than about 95 molar percent of sp²-hybridized carbon.

28. The method of claim 25,
wherein a resistivity of the turbostratic graphite is less than about 500 μOhm m.

29. The method of claim 25,
wherein the turbostratic graphite is polycrystalline.

30. The method of claim 25,
wherein the chemical vapor deposition is a plasma-enhanced chemical vapor deposition process.

31. The method of claim 25,
wherein the chemical vapor deposition is carried out at a temperature of less than about 500° C.

32. The method of claim 25,
wherein the annealing is carried out at a temperature greater than about 700° C.

33. The method of claim 25,
wherein the annealing comprises reducing a hydrogen content of the layer.

34. The method of claim 25,
wherein the layer is annealed at least one of after depositing the layer or during depositing the layer.

35. The method of claim 25, further comprising:
forming an electrode structure, the electrode structure electrically contacting the layer.

36. The method of claim 33, further comprising:
adjusting thickness and crystallite size and the hydrogen content of turbostratic graphite in the sensor layer to thereby influence a sensitivity of the sensor layer towards humidity, gases or biomolecules.
